(19) United States
(12) Patent Application Publication
(10) Pub. No.: US 2026/0139277 A1
(43) Pub. Date: May 21, 2026

(54) BASE EDITING APPROACHES FOR THE TREATMENT OF BETA-HEMOGLOBINOPATHIES

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR)

(72) Inventors: Annarita MICCIO, Paris (FR); Mathieu VON JOEST, Paris (FR)

(21) Appl. No.: 18/695,406

(22) PCT Filed: Sep. 27, 2022
(86) PCT No.: PCT/EP2022/076861

§ 371 Date: Mar. 26, 2024

(30) Foreign Application Priority Data
Sep. 28, 2021 (EP) 21306340.7

(51) Int. Cl. C12N 15/90; C12N 9/22; C12N 9/78; C12N 15/11

(52) U.S. Cl. CPC C12N 15/907; C12N 9/226; C12N 9/78; C12N 15/11; C12Y 305/04004; C12N 2310/20

(57) ABSTRACT

The clinical history of β-hemoglobinopathies shows that the severity is mitigated by the reduction of α-globin expression, resulting from co-inheritance of α-thalassemia. The inventors identified several mutations (T>C or A>G) that can disrupt binding motifs of transcription factors using CBE- and ABE-mediated base-editing approaches. In particular, the inventors designed gRNAs that, when combined with CBEs or ABEs, disrupt binding sites for transcriptional activators (GATA1 and NF-E2) in the MCS-R2 and recapitulate the beneficial α-globin reduction observed in patients presenting both β-hemoglobinopathies and α-thalassemia. Accordingly, the present invention relates to base editing approaches for the treatment of β-hemoglobinopathies.

### FIELD OF THE INVENTION

The present invention is in the field of medicine, in particular haematology.

### BACKGROUND OF THE INVENTION

β-hemoglobinopathies, β-thalassemia and sickle cell disease (SCD), are monogenic diseases caused by mutations in the β-globin locus, affecting the synthesis or the structure of the adult hemoglobin (Hb). β-thalassemia is caused by mutations in the β-globin gene (HBB) locus that reduce (β⁺) or abolish (β⁰) the production of β-globin chains included in the adult hemoglobin (HbA) tetramer, leading to the precipitation of uncoupled α-globin chains, erythroid cell death and severe anemia¹. In SCD, an A>T mutation in the HBB gene causes the substitution of valine for glutamic acid at position 6 of the β-globin chain (β^{S}), which is responsible for deoxygenation-induced polymerization of sickle hemoglobin (HbS). This primary event drives red blood cell (RBC) sickling, hemolysis, vaso-occlusive crises, multi-organ damage, often associated with severely reduced life expectancy².

The only definitive cure for β-hemoglobinopathies is transplantation of allogeneic hematopoietic stem cells (HSCs) from an HLA-compatible donor, an option available to <30% of the patients³. Gene therapy approaches based on the transplantation of autologous, genetically modified HSCs have been investigated as a treatment option for patients lacking a compatible donor⁴. Genome editing technology has been exploited to develop therapeutic approaches for β-hemoglobinopathies, based on direct gene correction. These approaches use designer nucleases, such as the CRISPR/Cas9 system that induces DNA double-strand breaks (DSBs) via a single guide RNA (gRNA) complementary to a specific genomic target⁴.

The clinical history of β-hemoglobinopathies shows that the severity of both β-thalassemia and SCD is mitigated by co-inheritance of α-thalassemia, which is characterized by the deletion of at least one copy of α-globin coding genes in HBA locus⁵⁻⁸. Deletions of one or multiple copies of HBA1 and/or HBA2 genes, which both encode α-globin, results in a decreased expression of α-globin. The presence of α-thalassemia traits in β-thalassemia patients improves the globin chain balance and reduces the excess of free α-chains^{5,9}. SCD patients with α-thalassemia display reduced anemia thanks to a decrease in RBC hemolysis^{6,8}.

HBA1 and HBA2 genes are under the control of a Locus Control Region (LCR) composed of 4 enhancers, named Multispecies Conserved Sequence 1 to 4 (MCS-R1 to MCS-R4) and located 10 to 50 kb upstream of HBA2 (FIG. 1)^{9,10}. Among them, MCS-R2, also known as HS-40, is located 40 kb upstream of HBA2 and is the most critical for α-globin expression. MCS-R2 is a 360 bp region (SEQ ID NO 1) containing a ≈260-bp core region (bold sequence) constituted of several activator binding sites, notably for GATA1 (underlined) and NF-E2 (double underlined) transcription factors (TFs)^{11,12}. Recent genome-editing strategies have been developed with the aim of partially decreasing α-globin expression as a potential therapy for β-thalassemia, based on the deletion of the entire MCS-R2 region in hematopoietic stem/progenitor cells (HSPCs)¹². CRISPR/Cas9 deletion of MCS-R2 via the use of a pair of gRNAs targeting DNA sequences surrounding the MCS-R2 region significantly downregulated the expression of α-globin. However, tie deletion of the whole region might cause an excessive reduction of α-globin, which in turn might lead to the generation of an α-thalassemic phenotype in SCD or β-thalassemic patients.

In addition, it is noteworthy that HSCs are highly sensitive to DNA DSBs¹³-especially in cases of multiple on-targets or concomitant on-target and off-target events. Even when highly specific gRNAs are used, Cas9/gRNA treatment of human HSPCs induces a DNA damage response that can lead to apoptosis¹⁴. CRISPR/Cas9 can cause P53-dependent cell toxicity and cell cycle arrest, resulting in the negative selection of cells with a functional P53 pathway¹⁵. Furthermore, the generation of several on-target DSBs, simultaneous on-target and off-target DSBs, or even a single on-target DSB is associated with a risk of deletion, inversion and translocation¹⁶. Hence, the development of novel, efficacious and safe therapeutic strategies for β-hemoglobinopathies based on precise DSB-free base editing rather than on DSB-induced DNA repair is preferred.

It has recently been shown that CRISPR-system-based cytosine and adenine base-editing enzymes (CBEs and ABEs) can make pinpoint changes in DNA with little or no DSB generation¹⁷. The basic components of base-editing enzymes are a catalytically disabled Cas9 nuclease and a deaminase; these eventually produce a C-G to T-A or A-T to G-C conversion (for CBEs and ABEs, respectively)¹⁸. Base-editing approaches allow precise DNA repair virtually in the absence of DSBs, and thus eliminate the risks of DSB-induced apoptosis, translocations and insertions or deletions of large portions of DNA. Furthermore, BEs have a lower level of off-target activity than Cas9 nuclease¹⁷. Importantly, base editing occurs in quiescent cells-suggesting that bona fide HSCs could be genetically modified using this novel technology¹⁹, and results in homogeneous, predictable base changes as compared to the heterogeneous and unpredictable mutagenesis induced by CRISPR/Cas9 nuclease.

### SUMMARY OF THE INVENTION

The present invention is defined by the claims. In particular, the present invention relates to base editing approaches for the treatment of β-hemoglobinopathies.

### DETAILED DESCRIPTION OF THE INVENTION

The clinical history of β-hemoglobinopathies shows that the severity is mitigated by the reduction of α-globin expression, resulting from co-inheritance of α-thalassemia. The inventors identified several mutations (T>C or A>G) that can disrupt binding motifs of transcription factors using CBE- and ABE-mediated base-editing approaches. In particular, the inventors designed gRNAs that, when combined with CBEs or ABEs, disrupt binding sites for transcriptional activators (GATA1 and NF-E2) and recapitulate the beneficial α-globin reduction observed in patients presenting both β-hemoglobinopathies and α-thalassemia. Accordingly, the present invention relates to base editing approaches for the treatment of β-hemoglobinopathies.

### Definitions

As used herein, the term “β-hemoglobinopathy” has its general meaning in the art and refers to any defect in the structure or function of any hemoglobin of an individual, and includes defects in the primary, secondary, tertiary or quaternary structure of hemoglobin caused by any mutation, such as deletion mutations or substitution mutations in the coding regions of the HBB gene, or mutations in, or deletions of, the promoters or enhancers of such gene that cause a reduction in the amount of hemoglobin produced as compared to a normal or standard condition.

As used herein, the term “sickle cell disease” has its general meaning in the art and refers to a group of autosomal recessive genetic blood disorders, which results from mutations in a globin gene and which is characterized by red blood cells that assume an abnormal, rigid, sickle shape. They are defined by the presence of βS-globin gene coding for a β-globin chain variant in which glutamic acid is substituted by valine at amino acid position 6 of the peptide: incorporation of the βS-globin in the Hb tetramers (HbS, sickle Hb) leads to Hb polymerization and to a clinical phenotype. The term includes sickle cell anemia (HbSS), sickle-hemoglobin C disease (HbSC), sickle beta-plus-thalassaemia (HbS/β+), or sickle beta-zerothalassaemia (HbS/β0).

As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains. As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin−CD34+CD38−CD90+CD45RA−, Lin−CD34+CD38−CD90−CD45RA−, Lin−CD34+CD38+IL−3aloCD45RA−, and Lin−CD34+CD38+CD10+ (Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “peripheral blood cells” refer to the cellular components of blood, including red blood cells, white blood cells, and platelets, which are found within the circulating pool of blood. In some embodiments, the eukaryotic cell is a bone marrow derived stem cell.

As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment.

As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-15 macrophage colony-stimulating factor (GM-CSF), stem cell factor (SCF), Fms-related tyrosine kinase 3 (flt-3) ligand, stromal cell-derived factor 1 (SDF-1); agonists of the chemokine (C-C motif) receptor 1 (CCR1), such as chemokine (C-C motif) ligand 3 (CCL3, also known as macrophage inflammatory protein-1α (Mip-1α)); agonists of the chemokine (C-X-C motif) receptor 1 (CXCR1) and 2 (CXCR2), such as chemokine (C-X-C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “isolated cell” refers to a cell that has been removed from an organism in which it was originally found, or a descendant of such a cell. Optionally the eukaryotic cell has been cultured in vitro, e.g., in the presence of other cells. Optionally the eukaryotic cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

As used herein, the term “alpha globin” or “α-globin” has its general meaning in the art and refers to protein that is encoded in human by the HBA1 and HBA2 genes. The human alpha globin gene cluster located on chromosome 16 spans about 30 kb and includes seven loci: 5′-zeta-pseudozeta-mu-pseudoalpha-1-alpha-2-alpha-1-theta-3′. The alpha-2 (HBA2) and alpha-1 (HBA1) coding sequences are identical. These genes differ slightly over the 5′ untranslated regions and the introns, but they differ significantly over the 3′ untranslated regions. The ENSEMBL IDs (i.e. the gene identifier number from the Ensembl Genome Browser database) for HBA1 and HBA2 are ENSG00000206172 and ENSG00000188536 respectively.

As used herein, the term “beta globin” or “β-globin”” has its general meaning in the art and refers to a globin protein, which along with alpha globin (HBA), makes up the most common form of haemoglobin (Hb) in adult humans. Normal adult human Hb is a heterotetramer consisting of two alpha chains and two beta chains. HBB is encoded by the HBB gene on human chromosome 11. It is 146 amino acids long and has a molecular weight of 15,867 Da.

As used herein, the term “gamma globin” or “γ-globin” has its general meaning in the art and refers to protein that is encoded in human by the HBG1 and HBG2 genes. The HBG1 and HBG2 genes are normally expressed in the fetal liver, spleen and bone marrow. Two γ-globin chains together with two α-globin chains constitute fetal hemoglobin (HbF) which is normally replaced by adult hemoglobin (HbA) in the year following birth. The ENSEMBL IDs (i.e. the gene identifier number from the Ensembl Genome Browser database) for HBG1 and HBG2 are ENSG00000213934 and ENSG00000196565 respectively.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the expression “repressing the expression of α-globin” indicates the expression of α-globin is at least 5% lower in the eukaryotic cell contacted with the gene editing platform of the present invention, than in a comparable eukaryotic cell that was not contacted with said gene editing platform. In some embodiments, the percentage of α-globin expression in the eukaryotic cell is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or less than an eukaryotic cell that was not contacted with the gene editing platform. In some embodiments, any method known in the art can be used to measure decrease in expression of α-globin (e. g. HPLC analysis of α-globin protein and RT-qPCR analysis of α-globin mRNA.) Typically, said methods are described in the EXAMPLE.

As used herein, the term “transcriptional activator” has its general meaning in the art and refers to a protein that increases gene transcription of a gene or set of genes. Most activators are DNA-binding proteins that bind to enhancers or promoter-proximal elements. According to the present disclosure, the activator is GATA1 or NF-E2.

As used herein, the term “NF-E2” has its general meaning in the art and refers to the Nuclear Factor, Erythroid 2 protein 45 kDa subunit. The term is also known as Leucine Zipper Protein NF-E2; p45; p45 NF-E2. NF-E2 is a TF that induces high-level expression of α-globin and other erythroid genes. The leucine zipper protein binds to a DNA sequence found in the LCR of α-globin, more precisely in the MCS-R2 region.

As used herein, the term “GATA1” has its general meaning in the art and refers to the GATA binding protein 1. The term is also known as GF1; GF-1; NFE1; XLTT; ERYF1; NF-E1; XLANP; XLTDA; and GATA-1. GATA1 is a protein that belongs to the GATA family of TFs. The protein plays an important role in erythroid development, modulating the expression of numerous genes expressed in erythroid cells. GATA1 binds to the MCS-R2 region.

As used herein, the term “transcriptional activator binding site” refers to a site present on DNA whereby the transcriptional activator according to the present disclosure binds. According to the present invention, the base-editing enzyme of the present invention edits the genome sequence of the eukaryotic cell so that the activator is able to bind to its transcriptional activator binding sites.

As used herein, the term “locus control region” or “LCR” has its general meaning the art and refers to a long-range cis-regulatory element that enhances expression of linked genes at distal chromatin sites. It functions in a copy number-dependent manner and is tissue-specific, as seen in the selective expression of α-globin genes in erythroid cells. The LCR functions by recruiting notably transcriptional activator. Its sequence is conserved in many vertebrates, and conservation of specific sites may suggest importance in function. It has been compared to a super-enhancer as both perform long-range cis regulation via recruitment of the transcription complex.

As used herein, the “MCS-R2 region” has its general meaning in the art and refers to a distal cis-acting enhancer located in the LCR of the HBA1 and HBA2 genes. MCS-R2, also known as HS-40, is located 40 kb upstream of HBA2 and is the most critical for α-globin expression. MCS-R2 is a 360 bp region containing a ≈260-bp core region constituted of several transcriptional activator binding sites, notably for GATA1 and NF-E2 transcription factors. An exemplary nucleic acid sequence for MCS-R2 is shown as SEQ ID NO:1. According to the present invention:
the “first GATA1 binding site” in the MCS-R2 region ranges from the nucleotide at position 17 to the nucleotide at position 20 in SEQ ID NO:1,
the “second GATA1 binding site” in the MCS-R2 region ranges from the nucleotide at position 112 to the nucleotide at position 115 in SEQ ID NO:1
the “third GATA1 binding site” in the MCS-R2 region ranges from the nucleotide at position 218 to the nucleotide at position 221 in SEQ ID NO:1
the “fourth GATA1 binding site” in the MCS-R2 region ranges from the nucleotide at position 253 to the nucleotide at position 256 in SEQ ID NO:1
the “first NF-E2 binding site” in the MCS-R2 region ranges from the nucleotide at position 129 to the nucleotide at position 135 in SEQ ID NO:1.
the “second NF-E2 binding site” in the MCS-R2 region ranges from the nucleotide at position 162 to the nucleotide at position 168 in SEQ ID NO:1.
SEQ ID NO 1: >Sequence of MCS-R2 enhancer. The 360 bp region containing a ≈260-bp core region is shown in bold sequence and is constituted of several activator binding sites, notably for GATA1 (underlined) and NF-E2 (double underlined) transcription factors (TFs)^{11,12}.

TCGACCCTCTGGAACCTATCAGGGACCACAGTCAGCCAGGCAAGCACATC
TGCCCAAGCCAAGGGTGGAGGCATGCAGCTGTGGGGGTCTGTGAAAACAC
TTGAGGGAGCAGATAACTGGGCCAACCATGACTCAGTGCTTCTGGAGGCC
AACAGGACTGCTGAGTCATCCTGTGGGGGTGGAGGTGGGACAAGGGAAAG
GGGTGAATGGTACTGCTGATTACAACCTCTGGTGCTGCCTCCCCCTCCTG
TTTATCTGAGAGGGAAGGCCATGCCCAAAGTGTTCACAGCCAGGCTTCAG
GGGCAAAGCCTGACCCAGACAGTAAATACGTTCTTCATCTGGAGCTGAAG
AAATTC

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “nucleic acid molecule” or “polynucleotide” refers to a DNA molecule (for example, but not limited to, a cDNA or genomic DNA). The nucleic acid molecule can be single-stranded or double-stranded.

As used herein, the term “isolated” when referring to nucleic acid molecules or polypeptides means that the nucleic acid molecule or the polypeptide is substantially free from at least one other component with which it is associated or found together in nature.

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, the term “hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

As used herein, the term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

As used herein the term “wild type” is a term of the art understood by skilled persons and means the typical form of an organism, strain, gene or characteristic as it occurs in nature as distinguished from mutant or variant forms.

As used herein, the term “derived from” refers to a process whereby a first component (e.g., a first molecule), or information from that first component, is used to isolate, derive or make a different second component (e.g., a second molecule that is different from the first).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the “linked” as used herein refers to the attachment of two or more entities to form one entity. A conjugate encompasses both peptide-small molecule conjugates as well as peptide-protein/peptide conjugates.

As used herein, the term “base-editing enzyme” refers to fusion protein comprising a defective CRISPR/Cas nuclease linked to a deaminase polypeptide. The term is also known as “base-editor”. Two classes of base-editing enzymes—cytosine base-editing enzymes (CBEs) and adenine base-editing enzymes (ABEs)—can be used to generate single base pair edits without double stranded breaks. Typically, cytosine base-editing enzymes are created by fusing the defective CRISPR/Cas nuclease to a deaminase.

As used herein, the term “deaminase” refers to an enzyme that catalyzes a deamination reaction. The term “deamination”, as used herein, refers to the removal of an amine group from one molecule. In In some embodiments, the deaminase is an adenosine deaminase

As used herein, the term “adenosine deaminase” has its general meaning in the art and refers to a polypeptide or fragment thereof capable of catalyzing the hydrolytic deamination of adenosine to inosine, which is treated like guanosine by the cell, creating an A to G (or T to C) conversion. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium.

As used herein, the term “nuclease” includes a protein (i.e. an enzyme) that induces a break in a nucleic acid sequence, e.g., a single or a double strand break in a double-stranded DNA sequence.

As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG for S. Pyogenes Cas9) protospacer adjacent motif (PAM) to specify the cut site (the 3^{rd} or the 4^{th} nucleotide upstream from PAM).

As used herein, the term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, the term “Cas9” refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1). Typically the Cas9 nuclease comprises the amino acid sequence as set forth in SEQ ID NO: 2.

SEQ ID NO: 2: Cas9 sequence
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKERGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDELDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD

As used herein, the term “defective CRISPR/Cas nuclease” refers to a CRISPR/Cas nuclease having lost at least one nuclease domain.

As used herein, the term “nickase” has its general meaning in the art and refers to an endonuclease which cleaves only a single strand of a DNA duplex. Accordingly, the term “Cas9 nickase” refers to a nickase derived from a Cas9 protein, typically by inactivating one nuclease domain of Cas9 protein.

As used herein, the term “guide RNA molecule” generally refers to an RNA molecule (or a group of RNA molecules collectively) that can bind to a Cas9 protein and target the Cas9 protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to using the CRISPR system as disclosed herein.

As used herein, the term “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “ribonucleoprotein complex,” or “ribonucleoprotein particle” refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue.

As used herein, the term “mutagenesis” refers to the introduction of mutations into a polynucleotide sequence. According to the present invention mutations are introduced into a target DNA molecule encoding for a variant domain of the antibody so as to mimic somatic hypermutation.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. A variant molecule can have entire sequence identity with the original parent molecule, or alternatively, can have less than 100% sequence identity with the parent molecule. For example, a variant of a sequence can be a second sequence that is at least 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% identical in sequence compare to the original sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is meant a sufficient amount of population of cells to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the cells with the desired specificity, on the age and weight of the recipient, and on the severity of the targeted condition. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 million of cells per kg. Usually 2 to 20 million of cells are injected in the subject. The desired purity can be achieved by introducing a sorting step. For uses provided herein, the cells are generally in a volume of a liter or less, can be 500 ml or less, even 250 ml or 100 ml or less. The clinically relevant number of cells can be apportioned into multiple infusions that cumulatively equal or exceed the desired total amount of cells.

### Methods for Repressing the Expression of α-Globin in a Eukaryotic Cell:

Accordingly, the first object of the present invention relates to a method for repressing the expression of α-globin in a eukaryotic cell comprising the step of contacting the eukaryotic cell with a gene editing platform that consists of a (a) at least one base-editing enzyme and (b) at least one guide RNA molecule for guiding the base-editing enzyme to at least one target sequence in the MCS-R2 region present in the locus control region of HBA1 and HBA2 genes, thereby editing said MCS-R2 region and subsequently repressing the expression of α-globin in said eukaryotic cell.

In some embodiments, the gene editing platform is suitable for introducing some mutations in the MCS-R2 region so that at least one transcriptional activator binding site is disrupted in said region. In some embodiments, the gene editing platform is particularly suitable for disrupting at least one transcriptional activator binding site for GATA1 or NF-E2 in the MCS-R2 region.

In some embodiments, the gene editing platform is particularly suitable for editing and disrupting the first, second, third and/or fourth GATA1 binding site(s) in the MCS-R2 region.

In some embodiments, the gene editing platform is particularly suitable for editing and disrupting the first GATA1 binding site (i.e. TATC sequence (SEQ ID NO: 21)) in the MCS-R2 region. In some embodiments, the gene editing platform is particularly suitable for introducing one A>G conversion in the first GATA1 binding site (i.e. TATC sequence SEQ ID NO: 21)).

In some embodiments, the gene editing platform is particularly suitable for editing and disrupting the second GATA1 binding site (i.e. GATA sequence SEQ ID NO: 22)) in the MCS-R2 region. In some embodiments, the gene editing platform is particularly suitable for introducing at least one A>G conversion in the second GATA1 binding site (i.e. GATA sequence SEQ ID NO: 22)).

In some embodiments, the gene editing platform is particularly suitable for editing and disrupting the third GATA1 binding site (i.e. GATT sequence (SEQ ID NO: 23)) in the MCS-R2 region. In some embodiments, the gene editing platform is particularly suitable for introducing at least one A>G conversion in the third GATA1 binding site (i.e. GATT sequence (SEQ ID NO: 23)).

In some embodiments, the gene editing platform is particularly suitable for editing and disrupting the fourth GATA1 binding site (i.e. TATC sequence (SEQ ID NO: 24)) in the MCS-R2 region. In some embodiments, the gene editing platform is particularly suitable for introducing at least one A>G conversion in the fourth GATA1 binding site (i.e. TATC sequence (SEQ ID NO: 24)).

In some embodiments, the gene editing platform is particularly suitable for editing and disrupting the first NF-E2 binding site (i.e. TGACTCA sequence (SEQ ID NO: 25)) in the MCS-R2 region. In some embodiments, the gene editing platform is particularly suitable for introducing at least one A>G conversion in the second NF-E2 binding site (i.e. TGACTCA sequence (SEQ ID NO: 25)).

In some embodiments, the eukaryotic cell is selected from the group consisting of hematopoietic progenitor cells, hematopoietic stem cells (HSCs), pluripotent cells (i.e. embryonic stem cells (ES) and induced pluripotent stem cells (iPS)). Typically, the eukaryotic cell results from a stem cell mobilization.

In some embodiments, the base-editing enzyme of the present invention comprises a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA molecule as defined hereinafter. However the defective CRISPR/Cas nuclease of the invention is a modified version with no nuclease activity. Accordingly, the defective CRISPR/Cas nuclease specifically recognizes the guide RNA molecule and thus guides the base-editing enzyme to its target DNA sequence.

In some embodiments, the defective CRISPR/Cas nuclease can be modified to increase nucleic acid binding affinity and/or specificity, alter an enzymatic activity, and/or change another property of the protein. In some embodiments, the nuclease domains of the protein can be modified, deleted, or inactivated. In some embodiments, the protein can be truncated to remove domains that are not essential for the function of the protein. In some embodiments, the protein is truncated or modified to optimize the activity of the RNA binding domain.

In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the mutant comprises an amino acid sequence having at least 50% of identity with the wild type amino acid sequence of the CRISPR/Cas nuclease. Various CRISPR/Cas nucleases can be used in this invention. Non-limiting examples of suitable CRISPR/CRISPR/Cas nucleases include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761 WO2014144592, WO2013176772, US20140273226, and US20140273233, the contents of which are incorporated herein by reference in their entireties.

In some embodiments, the CRISPR/Cas nuclease is derived from a type II CRISPR-Cas system. In some embodiments, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina, inter alia.

In some embodiments, the CRISPR/Cas nuclease is a mutant of a wild type CRISPR/Cas nuclease (such as Cas9) or a fragment thereof. In some embodiments, the CRISPR/Cas nuclease is a mutant Cas9 protein from S. pyogenes.

Methods for generating a Cas9 protein (or a fragment thereof) having an inactive DNA cleavage domain are known (See, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9 (Jinek et al., Science. 337:816-821 (2012); Qi et al., Cell. 28; 152 (5): 1173-83 (2013).

In some embodiments, the CRISPR/Cas nuclease of the present invention is nickase and more particularly a Cas9 nickase i.e. the Cas9 from S. pyogenes having one mutation selected from the group consisting of D10A and H840A. In some embodiments, the nickase of the present invention comprises the amino acid sequence as set forth in SEQ ID NO: 3 or SEQ ID NO:4.

SEQ ID NO: 3> S. pyogenes nCas9 Protein Sequence having the D10A
mutation
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDELDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD
SEQ ID NO: 4> S. pyogenes nCas9 Protein Sequence having the H840A
mutation
MDKKYSIGLDIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNEDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDELDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDAIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD

In some embodiments, the Cas9 variants having mutations other than D10A or H840A are used, which e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants of dCas9 are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 2 or 3. In some embodiments, variants of dCas9 are provided having amino acid sequences which are shorter, or longer than SEQ ID NO: 2 or 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

According to the present invention, the second component of the base-editing enzyme herein disclosed comprises a non-nuclease DNA modifying enzyme that is a deaminase.

In some embodiments, the deaminase is an adenosine deaminase. In some embodiments, the deaminase is an ADAT family deaminase. In some embodiments, the adenosine deaminase variant is a TadA deaminase. In some embodiments, the adenosine deaminase variant is a Staphylococcus aureus TadA, a Bacillus subtilis TadA, a Salmonella typhimurium TadA, a Shewanella putrefaciens TadA, a Haemophilus influenzae F3031 TadA, a Caulobacter crescentus TadA, or a Geobacter sulfurreducens TadA, or a fragment thereof. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20C-terminal amino acid residues relative to the full length ecTadA. In some embodiments, the TadA deaminase is TadA*7.10. In some embodiments, the TadA deaminase is a TadA*8 variant. For example, deaminase are described in International PCT Application WO2018/027078, WO2017/070632, WO/2020/168132, WO/2021/050571 each of which is incorporated herein by reference for its entirety. Also, see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3: eaao4774 (2017)), and Rees, H. A., et al., “Base editing: precision chemistry on the genome and transcriptome of living cells.” Nat Rev Genet. 2018 December; 19 (12): 770-788. doi: 10.1038/s41576-018-0059-1, the entire contents of which are hereby incorporated by reference. An exemplary amino acid sequence for the wild type TadA(wt) adenosine deaminase is shown as SEQ ID NO: 5. In some embodiments, the amino acid sequence of the adenosine deaminase comprises at least 90% sequence identity to SEQ ID NO:5. In some embodiments, the amino acid sequence of the adenosine deaminase comprises the modification at position 82 as numbered in SEQ ID NO: 5. In some embodiments, the amino acid sequence comprises of the adenosine deaminase comprises a V82S modification, wherein position 82 is as numbered in SEQ ID NO: 5. In some embodiments, the amino acid sequence of the adenosine deaminase comprises the modification at position 166 as numbered in SEQ ID NO:5. In some embodiments, the amino acid sequence of the adenosine deaminase comprises a T166R modification, wherein position 166 is as numbered in SEQ ID NO: 5. In some embodiments, the amino acid sequence of the adenosine deaminase comprises modifications at positions 82 and 166 as numbered in SEQ ID NO: 5. In some embodiments, the amino acid sequence of the adenosine deaminase comprises V82S and T166R modifications, wherein positions 82 and 166 are as numbered in SEQ ID NO: 5. In some embodiments, the adenosine deaminase variant further comprises one or more of the following alterations: Y147T, Y147R, Q154S, Y123H, and Q154R. In some embodiments, the adenosine deaminase variant comprises a combination of alterations selected from the group consisting of: Y147T+Q154R; Y147T+Q154S; Y147R+Q154S; V82S+Q154S; V82S+Y147R; V82S+Q154R; V82S+Y123H; 176Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and I76Y+V82S+Y123H+Y147R+Q154R. In some embodiments, the adenosine deaminase variant is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In some embodiments, the adenosine deaminase is provided as a single (e.g., provided as a monomer) TadA variant as described above. In some embodiments, adenosine deaminase is provided as a heterodimer of a wild-type TadA (TadA(wt)) linked to a TadA variant as described above.

SEQ ID NO: 5 > TadA sequence
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD

In some embodiments, the deaminase is fused to the N-terminus of the defective CRISPR/Cas nuclease. In some embodiments, the deaminase is fused to the C-terminus of the defective CRISPR/Cas nuclease. In some embodiments, the defective CRISPR/Cas nuclease and the deaminase are fused via a linker. In some embodiments, the linker comprises a (GGGGS)n (SEQ ID NO:6), a (G)n, an (EAAAK)n (SEQ ID NO: 7), a (GGS)n, an SGSETPGTSESATPES (SEQ ID NO: 8) motif (see, e.g., Guilinger J P, Thompson D B, Liu D R. Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65 (10): 1357-69, the entire contents of which are incorporated herein by reference.

In some embodiments, the fusion protein may comprise additional features. Other exemplary features that may be present are localization sequences, such as nuclear localization sequences (NLS), cytoplasmic localization sequences, export sequences, such as nuclear export sequences, or other localization sequences, as well as sequence tags that are useful for solubilization, purification, or detection of the fusion proteins. Suitable localization signal sequences and sequences of protein tags are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable features will be apparent to those of skill in the art.

Various base-editing enzymes are known in the art (see e.g. Improving cytidine and adenine base-editing enzymes by expression optimization and ancestral reconstruction. Nat Biotechnol. 2018 May 29) and typically include those described in Table A.

TABLE A
some exemplary base-editing enzymes

| Base-editing | |
|---|---|
| enzyme | References |
| ABEmax | Improving cytidine and adenine base-editing enzymes by expression |
| | optimization and ancestral reconstruction. Nat Biotechnol. 2018 May 29. |
| | pii: nbt.4172. doi: 10.1038/nbt.4172. |
| AncBE4max | Improving cytidine and adenine base-editing enzymes by expression |
| | optimization and ancestral reconstruction. Nat Biotechnol. 2018 May 29. |
| | pii: nbt.4172. doi: 10.1038/nbt.4172. |
| evoCDA1- | Continuous evolution of base-editing enzymes with expanded target |
| BE4max-NG | compatibility and improved activity. Nat Biotechnol. 2019 Jul. 22. pii: |
| | 10.1038/s41587-019-0193-0. doi: 10.1038/s41587-019-0193-0. |
| evoFERNY- | Continuous evolution of base-editing enzymes with expanded target |
| BE4max | compatibility and improved activity. Nat Biotechnol. 2019 Jul. 22. pii: |
| | 10.1038/s41587-019-0193-0. doi: 10.1038/s41587-019-0193-0. |
| CBE-NRCH | Miller S M, Wang T, Randolph P B, Arbab M, Shen M W, Huang T P, |
| | Matuszek Z, Newby G A, Rees H A, Liu D R. Continuous evolution of |
| | SpCas9 variants compatible with non-G PAMs. Nat Biotechnol. 2020 |
| | Apr.; 38(4): 471-481. doi: 10.1038/s41587-020-0412-8. Epub 2020 Feb. 10. |
| | PMID: 32042170; PMCID: PMC7145744. |
| CBE-SpG | Walton R T, Christie K A, Whittaker M N, Kleinstiver B P. Unconstrained |
| | genome targeting with near-PAMless engineered CRISPR-Cas9 variants. |
| | Science. 2020 Apr. 17; 368(6488): 290-296. doi: 10.1126/science.aba8853. |
| | Epub 2020 Mar. 26. PMID: 32217751; PMCID: PMC7297043. |
| CBE-SpRY | Walton R T, Christie K A, Whittaker M N, Kleinstiver B P. Unconstrained |
| | genome targeting with near-PAMless engineered CRISPR-Cas9 variants. |
| | Science. 2020 Apr. 17; 368(6488): 290-296. doi: 10.1126/science.aba8853. |
| | Epub 2020 Mar. 26. PMID: 32217751; PMCID: PMC7297043. |
| ABE-SpRY | Walton R T, Christie K A, Whittaker M N, Kleinstiver B P. Unconstrained |
| | genome targeting with near-PAMless engineered CRISPR-Cas9 variants. |
| | Science. 2020 Apr. 17; 368(6488): 290-296. doi: 10.1126/science.aba8853. |
| | Epub 2020 Mar. 26. PMID: 32217751; PMCID: PMC7297043. |
| ABE8e | Richter M F, Zhao K T, Eton E, Lapinaite A, Newby G A, Thuronyi B W, |
| | Wilson C, Koblan L W, Zeng J, Bauer D E, Doudna J A and Liu D R. Phage- |
| | assisted evolution of an adenine base editor with improved Cas domain |
| | compatibility and activity. Nat. Biotechnol. 2020 Mar. 38, 883-891. 1780 |
| | doi: 10.1038/s41587-020-0453-z |

The second component of the gene-editing platform disclosed herein consists of at least one guide RNA molecule suitable for guiding the base-editing enzyme to at least one target sequence located in the MCS-R2 region. The guide RNA molecule of the present invention thus comprises a guide sequence for providing the targeting specificity. It includes a region that is complementary and capable of hybridization to a pre-selected target site of interest in the MCS-R2 region (e.g. a GATA1 binding site or a NF-E2 binding site).

In some embodiment, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In some embodiments, the guide sequence is about 17-20 nucleotides in length, such as 20 nucleotides.

Typically, a software program is used to identify candidate CRISPR target sequences on both strands of the DNA nucleic acid molecule containing the HBG genes based on desired guide sequence length and a CRISPR motif sequence (PAM) for a specified CRISPR enzyme. One requirement for selecting a suitable target nucleic acid is that it has a 3′ PAM site/sequence. Each target sequence and its corresponding PAM site/sequence are referred herein as a Cas-targeted site. Type II CRISPR system, one of the most well characterized systems, needs only Cas 9 protein and a guide RNA complementary to a target sequence to affect target cleavage. For example, target sites for Cas9 from S. pyogenes, with PAM sequences NGG, may be identified by searching for 5′-Nx-NGG-3′ both on the input sequence and on the reverse-complement of the input. Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a “seed” sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed: PAM sequence in the relevant genome. The user may be allowed to choose the length of the seed sequence. The user may also be allowed to specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s). Further details of methods and algorithms to optimize sequence selection can be found in U.S. application Ser. No. 61/836,080; incorporated herein by reference.

The guide RNA molecule of the present invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U). In particular, the RNA molecule of the present invention can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties.

In some embodiments, the guide RNA molecule may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine.

In some embodiments, the base-editing enzyme and the corresponding guide RNA molecule is chosen according to Table B.

TABLE B
suitable pairings between base-editing enzymes and guide RNA molecule

| | Selected base- | | |
|---|---|---|---|
| Goal / Target | editing enzyme | Selected gRNA molecule | gRNA name |
| Disruption of | ABE-NRCH or | ACCUAUCAGGGACCACAGUC | gRNA1 |
| 1^{st} GATA1 | ABE-SpRY | (SEQ ID NO: 9) | |
| binding site | | | |
| Disruption of | ABE-NRCH or | CAGAUAACUGGGCCAACCAU | gRNA2 |
| 2^{nd} GATA1 | ABE-SpRY | (SEQ ID NO: 10) | |
| binding site | | | |
| Disruption of | ABE-NRTH | AGUUAUCUGCUCCCUCAAGU | gRNA3 |
| 2^{nd} GATA1 | | (SEQ ID NO: 11) | |
| binding site | | | |
| Disruption of | ABE-NRCH or | UUGUAAUCAGCAGUACCAUU | gRNA4 |
| 3^{rd} GATA1 | ABE-SpRY | (SEQ ID NO: 12) | |
| binding site | | | |
| Disruption of | ABEmax or | CUCAGAUAAACAGGAGGGGG | gRNA5 |
| 4^{th} GATA1 | ABE-SpRY | (SEQ ID NO: 13) | |
| binding site | | | |
| Disruption of | ABEmax or | CCAUGACUCAGUGCUUCUGG | gRNA6 |
| 1^{st} NF- | ABE SpRY | (SEQ ID NO: 14) | |
| E2 | | | |
| binding | | | |
| site | | | |
| Disruption of | ABE-NRTH or | UGAGUCAUGGUUGGCCCAGU | gRNA10 |
| 1^{st} NF- | ABE SpRY | (SEQ ID NO: 26) | |
| E2 | | | |
| binding | | | |
| site | | | |
| Disruption of | ABE SpRY | UGCUGAUUACAACCUCUGGU | gRNA11 |
| 3^{rd} | | (SEQ ID NO: 27) | |
| GATA1 | | | |
| binding | | | |
| site | | | |
| Disruption of | ABE SpRY | GCUGAUUACAACCUCUGGUG | gRNA12 |
| 3^{rd} | | (SEQ ID NO: 28) | |
| GATA1 | | | |
| binding | | | |
| site | | | |

In some embodiments, a plurality of guide RNA molecules are designed for targeting a plurality of sequences in the MCS-R2 region. In some embodiments, the gene editing platform disclosed herein thus comprises 2, 3, 4, 5, 6, 7, 8, 9, 10, 11, 12, 13, 14, 15, 16, 17, 18, or 20 guide RNA molecules as disclosed herein.

In some embodiments, a plurality of base-editing enzyme along with a plurality of guide RNA molecules are designed for targeting a plurality of sequences in the MCS-R2 region. In some embodiments, the gene editing platform disclosed herein thus comprises 2, 3 or 4 base-editing enzymes and 2, 3, 4, 5, 6, 7, 8, 9, 10, 11, 12, 13, 14, 15, 16, 17, 18, or 20 RNA molecules as disclosed herein.

In some embodiments, the different components of the gene editing platform of the present invention are provided to the eukaryotic cell through expression from one or more expression vectors. For example, the nucleic acids encoding the guide RNA molecule or the base-editing enzyme can be cloned into one or more vectors for introducing them into the eukaryotic cell. The vectors are typically prokaryotic vectors, e.g., plasmids, or shuttle vectors, or insect vectors, for storage or manipulation of the nucleic acid encoding the guide RNA molecule or the base-editing enzyme herein disclosed. Preferably, the nucleic acids are isolated and/or purified. Thus, the present invention provides recombinant constructs or vectors having sequences encoding one or more of the guide RNA molecule or base-editing enzymes described above. Examples of the constructs include a vector, such as a plasmid or viral vector, into which a nucleic acid sequence of the invention has been inserted, in a forward or reverse orientation. In some embodiments, the construct further includes regulatory sequences. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the eukaryotic cell to be transformed, transfected, or infected, the desired expression level, and the like. Large numbers of suitable vectors and promoters are known to those of skill in the art, and are commercially available. Appropriate cloning and expression vectors for use with eukaryotic hosts are also described in e.g., Sambrook et al. (2001, Molecular Cloning: A Laboratory Manual, Cold Spring Harbor Press). The vector can be capable of autonomous replication or integration into a host DNA. The vector may also include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli. Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

In some embodiments, the different components of the gene editing platform of the present invention are provided to the population of cells through the use of an RNA-encoded system. For instance, the base-editing system may be provided to the population of cells through the use of a chemically modified mRNA-encoded adenine or cytidine base editor together with modified guide RNA as described in Jiang, T., Henderson, J. M., Coote, K. et al. Chemical modifications of adenine base editor mRNA and guide RNA expand its application scope. Nat Commun 11, 1979 (2020). In particular, engineered RNA-encoded base-editing enzymes (e.g. ABE) system are prepared by introducing various chemical modifications to both mRNA that encoded the base-editing enzyme and guide RNA. In particular said modifications consist in uridine depleted mRNAs modified with 5-methoxyuridine: synonymous codons may be introduced to deplete uridines as much as possible without altering the coding sequence and replaced all the remaining uridines with 5-methoxyuridine. Said optimized base editing system exhibits higher editing efficiency at some genomic sites compared to DNA-encoded system. It is also possible to encapsulate the modified mRNA and guide RNA into lipid nanoparticle (LNP) for allowing lipid nanoparticle (LNP)-mediated delivery.

In some embodiments, the different components of the gene editing platform of the present invention are provided to the population of cells through the use of ribonucleoprotein (RNP) complexes. For instance the base-editing enzyme can be pre-complexed with one or more guide RNA molecules to form a ribonucleoprotein (RNP) complex. The RNP complex can thus be introduced into the eukaryotic cell. Introduction of the RNP complex can be timed. The cell can be synchronized with other cells at G1, S, and/or M phases of the cell cycle. RNP delivery avoids many of the pitfalls associated with mRNA, DNA, or viral delivery. Typically, the RNP complex is produced simply by mixing the proteins (i.e. the base-editing enzyme) and one or more guide RNA molecules in an appropriate buffer. This mixture is incubated for 5-10 min at room temperature before electroporation. Electroporation is a delivery technique in which an electrical field is applied to one or more cells in order to increase the permeability of the cell membrane. In some embodiments, genome editing efficiency can be improved by adding a transfection enhancer oligonucleotide.

In some embodiments, a plurality of successive transfections are performed for reaching a desired level of mutagenesis in the cell.

### Methods of Therapy:

A further object of the present invention relates to a method of treating a β-hemoglobinopathy in a subject in need thereof, the method comprising transplanting a therapeutically effective amount of a population of eukaryotic cells obtained by the method as above described.

In some embodiments, the population of cell is autologous to the subject, meaning the population of cells is derived from the same subject.

In some embodiments, the β-hemoglobinopathy is a sickle cell disease.

In some embodiments, the β-hemoglobinopathy is a β-thalassemia.

### Kits

This invention further provides kits containing reagents for performing the above-described methods, including all component of the gene editing platform as disclosed herein for performing mutagenesis. To that end, one or more of the reaction components, e.g., guide RNA molecules, and nucleic acid molecules encoding for the base-editing enzymes for the methods disclosed herein can be supplied in the form of a kit for use. In some embodiments, the kit comprises one or more base-editing enzymes and one or more guide RNA molecules. In some embodiments, the kit can include one or more other reaction components. In some embodiments, an appropriate amount of one or more reaction components is provided in one or more containers or held on a substrate. Examples of additional components of the kits include, but are not limited to, one or more host cells, one or more reagents for introducing foreign nucleotide sequences into host cells, one or more reagents (e.g., probes or PCR primers) for detecting expression of the guide RNA or base-editing enzymes or verifying the target nucleic acid's status, and buffers or culture media for the reactions. The kit may also include one or more of the following components: supports, terminating, modifying or digestion reagents, osmolytes, and an apparatus for detection. The components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below −20° C., or otherwise in a frozen state. The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

The invention will be further illustrated by the following figures and examples. However, these examples and figures should not be interpreted in any way as limiting the scope of the present invention.

### FIGURES

FIG. 1: α-globin locus structure. Schematic representation of the α-globin locus on chromosome 16, depicting the HBA2 and HBA1 genes and their promoters and enhancers. MCS-R2 enhancer region: Schematic representation of MCS-R2 composition on chromosome 16. GATA1 BSs are depicted in dark grey and NF-E2 BSs are depicted in light grey. gRNA targeting the different BSs are in black.

FIG. 2: K562 were transfected with plasmids encoding BEs and gRNAs. A gRNA disrupting the α-globin coding sequence was used as control (gRNA KO) as well as gRNAs generating the MCS-R2 deletion, together with Cas9 nuclease. (A) Base editing efficiency, calculated by the EditR software in samples subjected to Sanger sequencing. The base editing efficiency percentage was measured by subtracting the percentage of the base conversion in the control that was considered as background noise. Number indicate the position of the nucleotide change according to the protospacer sequence (+1 is the 5′ nucleotide of the protospacer, i.e +21 for the PAM). Data are expressed as mean±SD (n=3 biologically independent experiments). Representative nucleotide change in binding sites are depicted using Rstudio. Nucleotide letter size are proportional to the base editing efficiency observed. (B) InDel frequency, calculated by the TIDE software in samples subjected to Sanger sequencing. (C) Deletion frequency was quantified by multiplexed digital droplet PCR. Cells transfected only with Cas9 were used as control. (D) α-globin expression was measured by RT-qPCR. We calculated the fold change compared to unedited controls transfected only with BEs (Ctrl). Data are expressed as mean±SD (n=3 biologically independent experiments).

FIG. 3: SCD HSPCs were transfected with BE mRNA and chemically modified gRNAs. A gRNA disrupting the α-globin coding sequence was used as control (gRNA KO) as well as gRNAs generating the MCS-R2 deletion. These latter gRNAs were coupled with Cas9 protein to form RNP complexes. (A) CFC frequency for control and edited samples. Data are expressed as mean±SD (n=3 biologically independent experiments). (B) Base editing efficiency, calculated by the EditR software in samples subjected to Sanger sequencing. The base editing efficiency percentage was measured by subtracting the percentage of the base conversion in the control that was considered as background noise. Number indicate the position of the nucleotide change according to the protospacer sequence (+1 is the 5′ nucleotide of the protospacer, i.e +21 for the PAM). Data are expressed as mean±SD (n=3 biologically independent experiments). Representative nucleotide change in binding sites are depicted using Rstudio. Nucleotide letter size are proportional to the base editing efficiency observed. (C) InDel frequency, calculated by the TIDE software in samples subjected to Sanger sequencing. (D) Deletion frequency, quantified by multiplexed digital droplet PCR. Cells transfected only with Cas9 and Cas9 with a control gRNA targeting the unrelated locus AAVS1 were used as control. (n=1 biologically independent experiment).

FIG. 4: (A) α-globin expression was measured by RT-qPCR in control and edited SCD BFU-E. We calculated the fold change compared to unedited controls transfected only with BEs and BEs with control gRNA targeting the unrelated locus AAVS1 (Ctrl). Data are expressed as mean±SD (n=3 biologically independent experiments). (B) α-globin chain expression was measured by Western Blot in control and edited SCD BFU-E. α-globin levels were normalized to β-actin. We calculated the fold change compared to unedited controls transfected only with BEs (Ctrl). Data are expressed as mean±SD (n=3 biologically independent experiments). (C) Expression of globin chains and hemoglobins was evaluated by RP-HPLC and CE-HPLC, respectively, in control and edited SCD BFU-E. Representative RP-HPLC and CE-HPLC chromatogram are shown. We calculated the percentage of each Hb type over the total Hb tetramers. Data are expressed as mean±SD (n=3 biologically independent experiments).

### EXAMPLE

### Methods

### Cell Line Culture

K562 were maintained in RPMI 1640 (Lonza) containing glutamine and supplemented with 10% fetal bovine serum (Lonza), 2 mM Hepes (Life Technologies), 100 nM sodium pyruvate (Life Technologies), and penicillin and streptomycin (Life Technologies).

### Plasmids

Plasmids used in this study include pCMV_ABEmax_P2A_GFP (Addgene #112101), pCMV-ABE4max-NRCH (Addgene #136923), ABEmax-NRTH (Addgene #136922), CBE-NRRH (Addgene #136918), pCMV-T7-ABEmax (7.10)-SpRY-P2A-EGFP (RTW5025) (Addgene #140003) and pMJ920 (Addgene #42234). The ABE-SpRY-OPT plasmid was created by inserting the 3′UTR+poly-A fragment in pCMV-T7-ABEmax (7.10)-SpRY-P2A-EGFP (RTW5025) (Addgene #140003).

gRNA Design

For the gRNA expression plasmid construction, oligonucleotides were annealed to create the gRNA protospacer and the duplexes were ligated into Bbs I-digested MA128 plasmid (provided by M. Amendola, Genethon, France).

### Plasmid Transfection

K562 (10⁶ cells/condition) were transfected with 3.6 μg of a base editing enzyme-expressing plasmid and 1.2 μg of gRNA-containing plasmid. For base editing enzyme plasmids that do not express GFP, we co-transfected 250 ng of a GFPmax expressing plasmid (Lonza). We used AMAXA Cell Line Nucleofector Kit V (VCA-1003) and U-16 program (Nucleofector II). 18 h after transfection, cells were sorted using SONY SH800 according to GFP expression. GFP+ cells were collected and maintained in culture for at least 3 days and at day 3 genomic DNA and RNA extraction were performed.

### Evaluation of Editing Efficiency

Genomic DNA was extracted from control and edited cells using PURE LINK Genomic DNA Mini kit (LifeTechnologies) following manufacturer's instructions, 4 days post-transfection. To evaluate editing efficiency at gRNA target sites, we performed PCR followed by Sanger sequencing and EditR (EditR: A Method to Quantify Base Editing from Sanger Sequencing)²¹ or TIDE analysis²².

### Primer Used:

Forward
(SEQ ID NO: 15)
5′-TGGTCCTGAAGGATGAGAAG-3′
Reverse
(SEQ ID NO: 16)
5′-AGCAACAGTCCTTTCTCTGG-3′

Digital Droplet PCR (ddPCR) was performed using EvaGreen mix to quantify the frequency of the 250-bp deletion (MCS-R2 region). Genomic DNA was amplified with two primer sets: one set with a primer within the deleted region and a primer upstream the deletion and the other set of primers located both 3.9 kb downstream of the deletion.

### Amplified Regions:

### MCS-R2:

Primer in the deleted region:
(SEQ ID NO: 29)
5′-GGCCCAGTTATCTGCTC CCTCAAGT-3′
Primer upstream of the deletion:
(SEQ ID NO: 30)
5′-AGGCCCATATCTCTGCCCAAGAGC-3′

### Control Region 3.9 kb Downstream of the Deletion:

Forward
(SEQ ID NO: 31)
5′-GGGGACTTTTGCCATGCCTGAAGTAGA-3′
Reverse
(SEQ ID NO: 32)
5′-GGCCCCACTCCCTGATCTTAACCATTT-3′

### RT-qPCR Analysis of Globin Transcripts

Total RNA was extracted from K562 using RNeasy micro kit (QIAGEN), following manufacturer's instructions. Mature transcripts were reverse-transcribed using SuperScript First-Strand Synthesis System for RT-qPCR (Invitrogen) with oligo (dT) primers. RT-qPCR was performed using iTaq universal SYBR Green master mix (Biorad) and a CFX 384 Touch Real-Time PCR Detection System (Biorad).

### Primer Used:

HBA1/2:
Forward
(SEQ ID NO: 17)
5′-CGGTCAACTTCAAGCTCCTAA-3′
Reverse
(SEQ ID NO: 18)
5′-ACAGAAGCCAGGAACTTGTC-3′
HBB:
Forward
(SEQ ID NO: 33)
5′-GCAAGGTGAACGTGGATGAAGT-3′
Reverse
(SEQ ID NO: 34)
5′-TAACAGCATCAGGAGTGGACAGA-3′
HBG:
Forward
(SEQ ID NO: 35)
5′-CCTGTCCTCTGCCTCTGCC-3′
Reverse
(SEQ ID NO: 36)
5′-GGATTGCCAAAACGGTCAC-3′
GAPDH:
Forward
(SEQ ID NO: 19)
5′-GAAGGTGAAGGTCGGAGT-3′
Reverse
(SEQ ID NO: 20)
5′-GAAGATGGTGATGGGATTTC-3′

### HSPC Purification and Culture

We obtained human non-mobilized peripheral blood CD34⁺ HSPCs from SCD patients. SCD samples eligible for research purposes were obtained from the “Hôpital Necker-Enfants malades” Hospital (Paris, France). Written informed consent was obtained from all adult subjects. All experiments were performed in accordance with the Declaration of Helsinki. The study was approved by the regional investigational review board (reference: DC 2014-2272, CPP Ile-de-France II “Hôpital Necker-Enfants malades”). HSPCs were purified by immunomagnetic selection with AutoMACS (Miltenyi Biotec) after immunostaining with the CD34 MicroBead Kit (Miltenyi Biotec). Forty-eight hours before transfection, CD34⁺ cells were thawed and cultured at a concentration of 5×10⁵ cells/ml in the “HSPC medium” containing StemSpan (STEMCELL Technologies) supplemented with penicillin/streptomycin (Gibco), 250 nM StemRegenin1 (STEMCELL Technologies), and the following recombinant human cytokines (PeproTech): human stem cell factor (SCF) (300 ng/ml), Flt-3L (300 ng/ml), thrombopoietin (TPO) (100 ng/ml), and interleukin-3 (IL-3) (60 ng/ml).

mRNA In Vitro Transcription

10 μg of BE-expressing plasmids (pCMV_ABEmax_P2A_GFP (Addgene #112101), pCMV-ABE4max-NRCH (Addgene #136923) or ABE-SpRY-OPT) were digested overnight with 20 Units of a restriction enzyme that cleaves once after the poly-A tail or after the stop codon, for constructs with or without a poly-A tail, respectively. The linearized plasmids were purified using a PCR purification kit (QIAGEN) and were eluted in 30 μl of DNase/RNase-free water. 1 μg of linearized plasmid was used as template for the in vitro transcription (ivt) reaction (MEGAscript, Ambion). The ivt protocol was modified as follows. The GTP nucleotide solution was used at a final concentration of 3.0 mM instead of 7.5 mM and the anti-reverse cap analog N7-Methyl-3′-O-Methyl-Guanosine-5′-Triphosphate-5′-Guanosine (ARCA, Trilink) was used at a final concentration of 12.0 mM resulting in a final ratio of Cap:GTP of 4:1 that allows efficient capping of the mRNA. The incubation time for the ivt reaction was reduced to 30 minutes. For constructs without a poly-A tail already included in the plasmid, an additional step of polyadenylation was performed using manufacturer's guidelines (Poly-A tailing kit, Ambion). mRNA was precipitated using lithium chloride and resuspended in TE buffer in a final volume that allowed to achieve a concentration of >1 μg/μl. The mRNA quality was evaluated using Bioanalyzer (Agilent).

### RNA Transfection

10⁵ to 2×10⁵ CD34⁺ HSPCs per condition were transfected with 3.0 μg of the enzyme encoding mRNA, respectively, and a synthetic gRNA at a final concentration of 2.3 μM. We used the P3 Primary Cell 4D-Nucleofector X Kit S or L (Lonza) and the CA137 program (Nucleofector 4D). Untransfected cells or cells transfected with TE buffer or with the enzyme-encoding mRNA only, or with the enzyme-encoding mRNA and a gRNA targeting the AAVS1 locus, served as negative controls.

### Ribonucleoprotein (RNP) Transfection

RNP complexes were assembled at room temperature using a 90 μM Cas9-GFP protein and a 180 μM synthetic gRNA (ratio Cas9:gRNA of 1:2). To delete MCS-R2, 125 μM Cas9-GFP protein and 125 μM of each synthetic gRNA were used (ratio Cas9:gRNAtotal of 1:2). CD34 HSPCs (10⁵ to 2×10⁵ cells/condition) were transfected with RNP complexes using the P3 Primary Cell 4D-Nucleofector X Kit S (Lonza) and the CA137 program (Nucleofector 4D) in the presence of a transfection enhancer (IDT). Untransfected cells or cells transfected with TE buffer or with the Cas9 only, or with Cas9 and a gRNA targeting the AAVS1 locus, served as negative controls.

### Colony-Forming Cell (CFC) Assay

CD34⁺ HSPCs were plated at a concentration of 1×10³ cells/mL in a methylcellulose-based medium (GFH4435, Stem Cell Technologies) under conditions supporting erythroid and granulocyte/monocyte differentiation. BFU-E and CFU-GM colonies were counted after 14 days. Colonies were randomly picked and collected as bulk populations (containing at least 25 colonies) to evaluate base-editing efficiency, globin expression by RT-qPCR and RP-HPLC and hemoglobin expression by CE-HPLC.

### RP-HPLC Analysis of Globin Chains

RP-HPLC analysis was performed using a NexeraX2 SIL-30AC chromatograph and the LC Solution software (Shimadzu). A 250×4.6 mm, 3.6 μm Aeris Widepore column (Phenomenex) was used to separate globin chains by HPLC. Samples were eluted with a gradient mixture of solution B A (water/acetonitrile/trifluoroacetic acid, 95:5:0.1) and solution (water/acetonitrile/trifluoroacetic acid, 5:95:0.1). The absorbance was measured at 220 nm.

### CE-HPLC Analysis of Hemoglobin Tetramers

Cation-exchange HPLC analysis was performed using a NexeraX2 SIL-30AC chromatograph and the LC Solution software (Shimadzu). A 2 cation-exchange column (PolyCAT A, PolyLC, Columbia, MD) was used to separate hemoglobin tetramers by HPLC. Samples were eluted with a gradient mixture of solution A (20 mM bis Tris, 2 mM KCN, pH=6.5) and solution B (20 mM bis Tris, 2 mM KCN, 250 mM NaCl, pH=6.8). The absorbance was measured at 415 nm.

### Western Blot

BFU-E were lysed for 30 min at 4° C. using a lysis buffer containing: 10 mM Tris, 1 mM EDTA, 0.5 mM EGTA, 1% Triton X-100, 0.1% SDS, 0.1% Na-deoxicholate, 140 mM NaCl (Sigma-Aldrich) and a protease inhibitor cocktail (Roche-Diagnostics). Cell lysates underwent 3 cycles of freezing/thawing (3 min at −80° C./3 min at 37° C.). After centrifugation, the supernatant was collected and protein concentration was measured using the Pierce™ BCA Protein Assay Kit (ThermoScientific). After electrophoresis and protein transfer, β-actin and α-globins were detected using the antibodies MAB1501 (Merck Millipore) and sc-31110 (SantaCruz), respectively. The bands corresponding to the different proteins were quantified using the Chemidoc and the Image lab Software (BioRad).

### Results

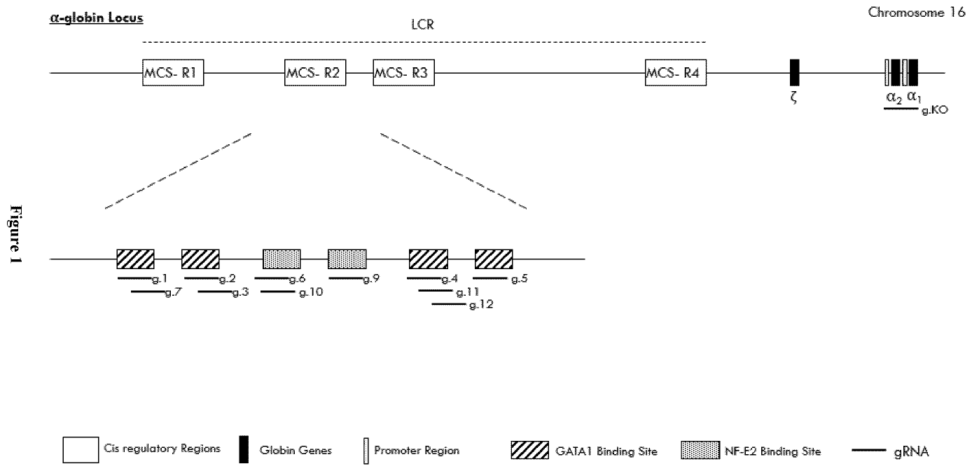

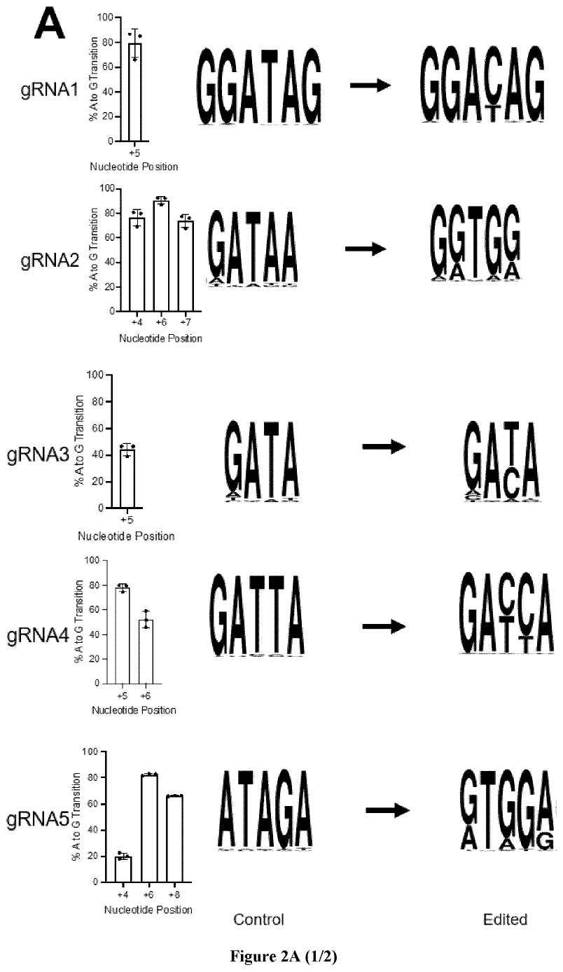

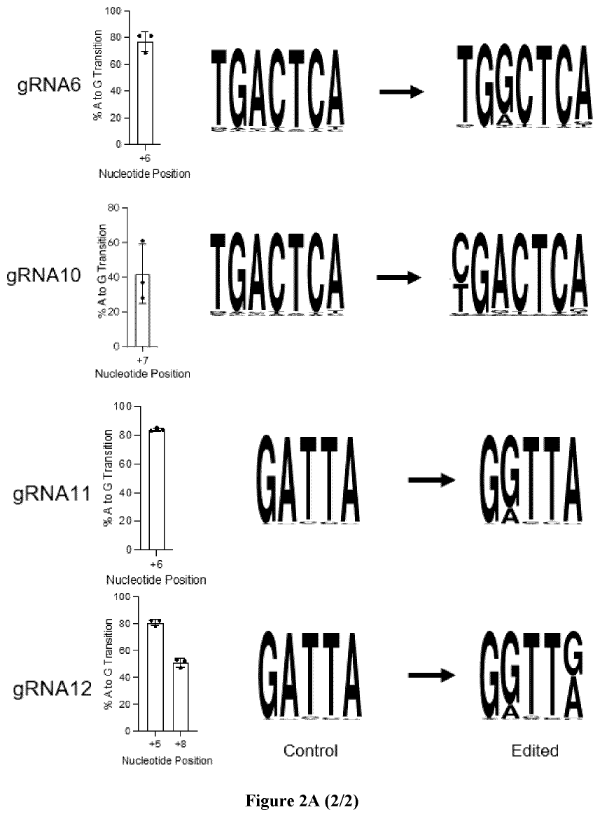

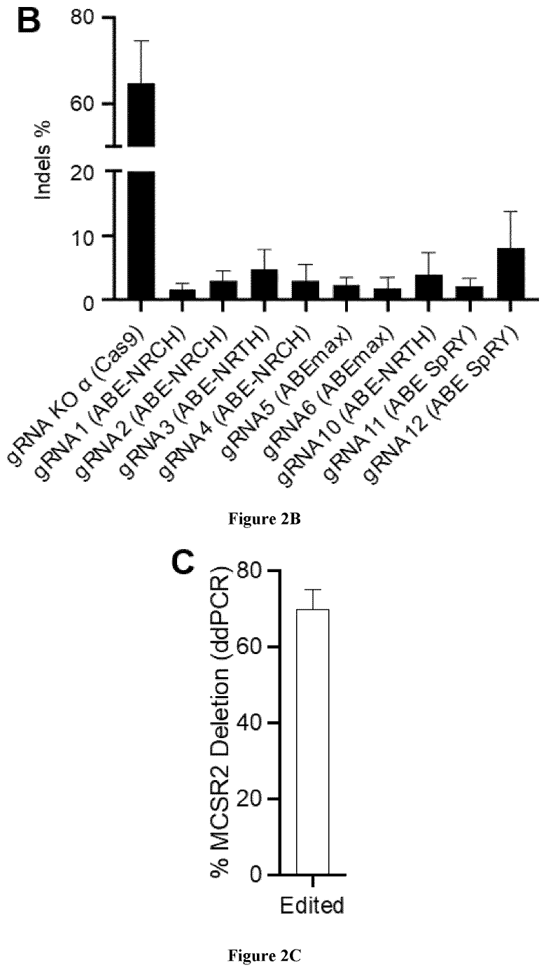

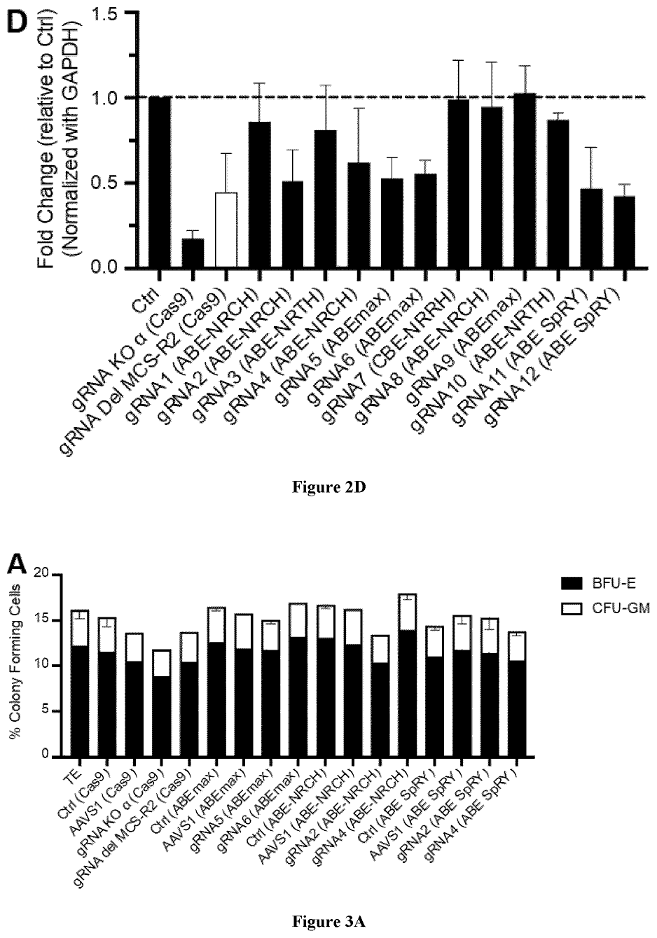

The inventors identified several mutations or regions in TF binding motifs located in MCS-R2 enhancer, which enable modulation of α-globin expression. (FIG. 1). Compared with a CRISPR/Cas9-nuclease-based strategy, a base-editing approach might allow the simultaneous targeting of multiple regions in the MCS-R2 region, e.g., GATA1 and NF-E2 binding motifs, or in different regions, e.g. GATA1 or NF-2 binding motifs in the MCS-R2 region and a region influencing fetal γ-globin expression to increase the levels of therapeutic fetal hemoglobin (e.g., γ-globin promoters in chromosome 11 to create binding sites for transcriptional activators or disrupt binding sites for transcriptional repressors or enhancers of the BCL11A gene on chromosome 2 encoding a transcriptional γ-globin repressor). Indeed, a Cas9-nuclease-based strategy targeting two different regions of the α-globin enhancers would probably trigger the deletion of the intervening sequence, which may be critical for enhancer activity. Similarly, the simultaneous cleavage in the MCS-R2 region and the γ-globin promoters or BCL11A enhancers could lead to chromosomal translocations. Thus, the inventors designed gRNAs that, when combined with ABEs (Table A), disrupt transcriptional activator binding sites (BSs) for different TFs in MCS-R2 region (FIG. 1). In particular, MCS-R2 region contains 4 GATA1 binding sites and 2 NF-E2 binding sites. gRNAs against these different regions have been designed (Table B) and combined with different ABEs enzymes for evaluation (Table A) in K562 erythroid cell line. A gRNA allowing Cas9 nuclease-mediated disruption of the α-globin coding sequence was used as control (gRNA KO)²⁰. This gRNA KO targets the first exon of HBA1 and HBA2. We also used as control Cas9 nuclease and two gRNAs generating the MCS-R2 deletion (gRNA Del MCS-R2). We transfected K562 cells with: (i) plasmids coding for ABE-NRCH, gRNA 1, 2, 4 or 8 and a GFPmax-expressing plasmid (ii) plasmids coding for ABEmax_GFP and gRNA 5, 6 or 9, (iii) plasmids coding for ABE-NRTH, gRNA 3 or 10 and a GFPmax-expressing plasmid, (iv) plasmids coding for CBE-NRRH, gRNA 7 and a GFPmax-expressing plasmid and (v) plasmids coding for ABE-SpRY, gRNA 11 or gRNA 12. After FACS sorting based on GFP expression, GFP+ cells were expanded and analyzed for base editing efficiency and HBA expression. Each gRNA with ABE editors successfully converted at least one adenine contained in GATA1 or NF-E2 DNA binding sequence (FIG. 2A). Combination of ABE-NRCH and gRNA1 resulted in one A>G conversion in 1^{st} GATA1 BS with an efficiency of 79%. Transfection with ABE-NRCH and gRNA2 successfully led to three A>G conversions in 2nd GATA1 BS with an efficiency ranging from 73% to 90%. Using ABE-NRTH and gRNA3, we obtained one A>G conversion in 2^{nd} GATA1 BS (44% efficiency). Two A>G conversions were observed with ABE-NRCH and gRNA4 in the 3rd GATA1 BS (78% and 52%, respectively) or with ABE-SpRY and gRNA12 (80% and 50% efficiency respectively) and one A>G conversion with ABE-SpRY and gRNA11 (84% efficiency). Using ABEmax and gRNA5, we obtained 3 A>G conversions in 4^{th} GATA1 BS (19%, 82 and 66% respectively). We generated one A>G conversion in 1st NF-E2 BS with ABEmax and gRNA6 combination (77% efficiency) or with the ABE-NRTH and gRNA 10 combination (42% efficiency) (FIG. 2A). Importantly, we did not observe DSB-induced insertion and deletions (InDels) (FIG. 2B). Using these 9 gRNAs, we were able to disrupt different TF binding sites involved in α-globin expression. Disruption of these different BSs efficiently diminished α-globin expression from 15% (gRNA1 and gRNA 10) to ˜40-60% (49% for gRNA2, 47% for gRNA5, and 45% for gRNA6, 52% for gRNA 11 and 57% for gRNA 12) (FIG. 2D). gRNA3 and gRNA4 diminished expression of α-globin by 19% and 37% respectively (FIG. 2D). In comparison, gRNA KO and MCS-R2 deletion reduced expression to 82% and 57%, respectively. The frequency of MCS-R2 deletion was ˜70% (FIG. 2C). Noteworthily, several gRNAs (i.e., gRNA7, gRNA8 and gRNA9) targeting the different TF BSs failed to diminish «-globin expression despite the efficient base conversion (FIG. 2D).

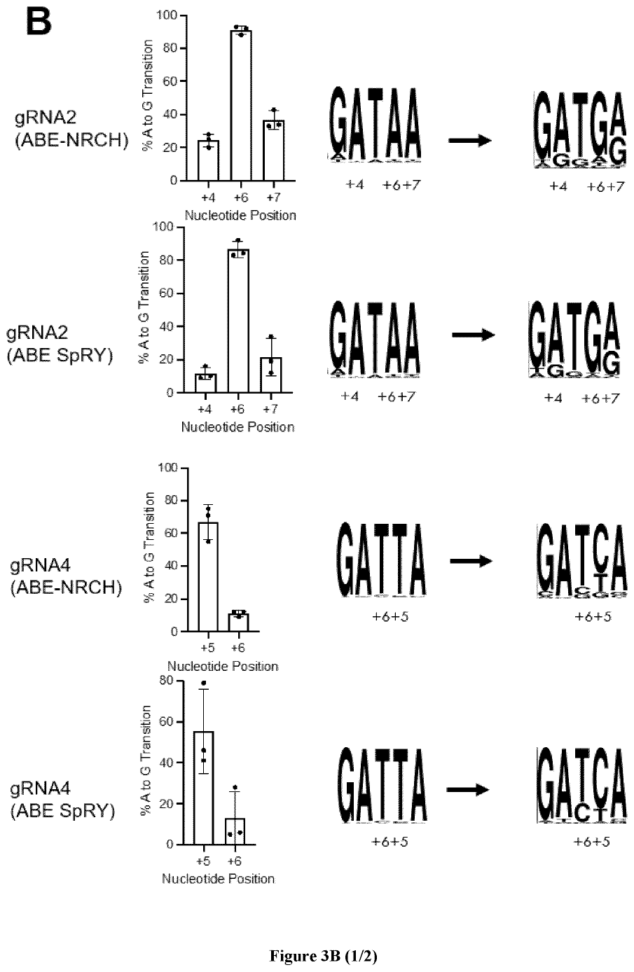

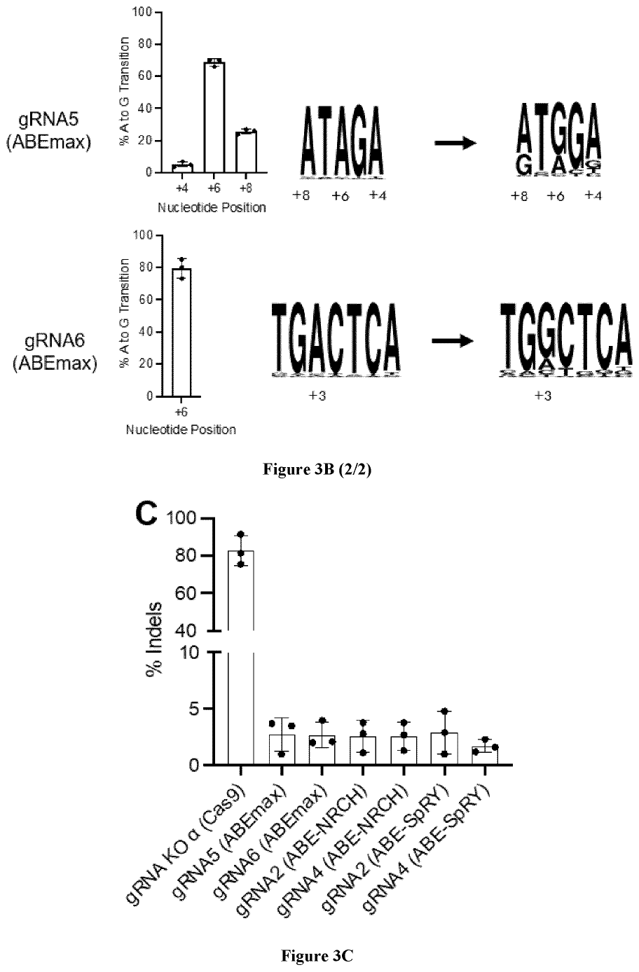

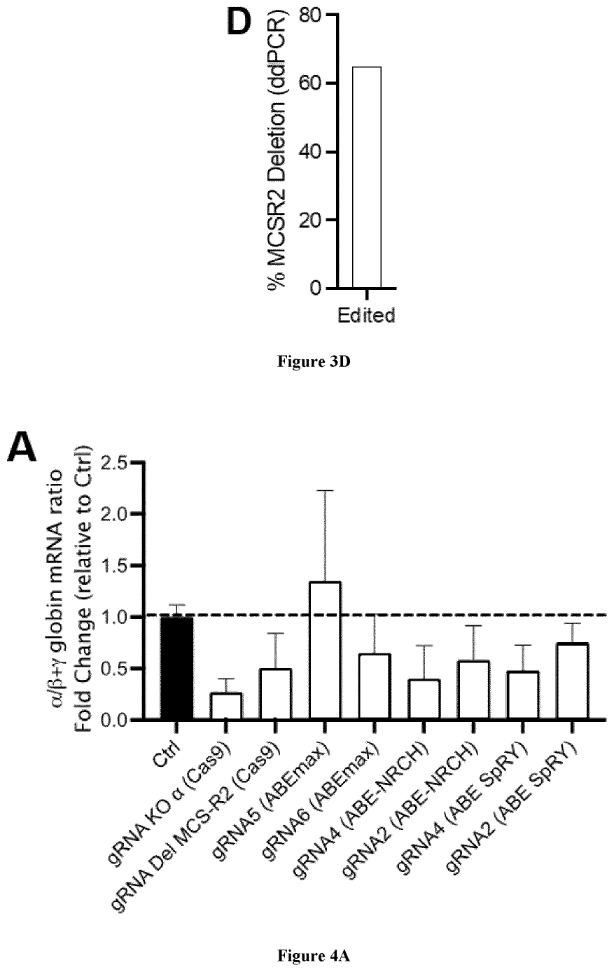

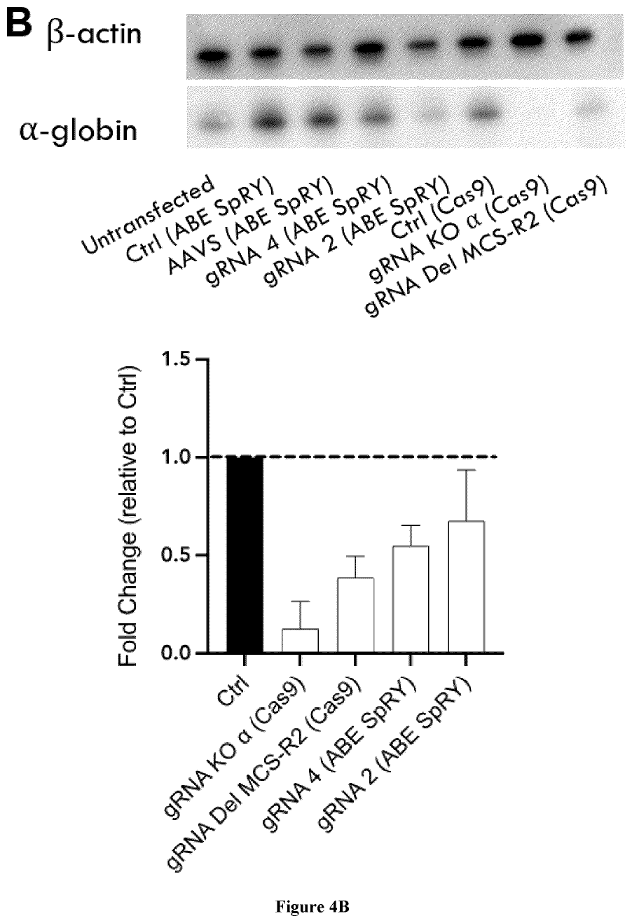

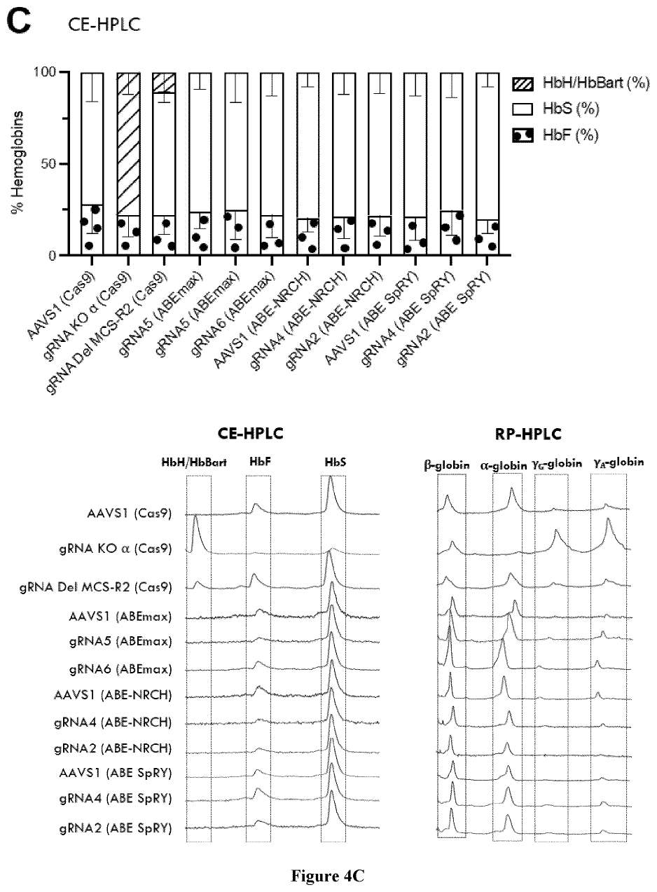

We then tested selected ABE/gRNA combinations in primary HSPCs from SCD patients. In particular we used gRNA 2 and 4 in combination with either ABE-NRCH or ABE-SpRY and gRNA 5 and 6 in combination with ABEmax. Controls include cells treated with ribonucleoprotein (RNP) complexes containing either the gRNA disrupting the α-globin coding sequence (gRNA KO) or the gRNAs generating the MCS-R2 deletion (gRNA Del MCS-R2). Cell were then plated in a semi-solid medium allowing the growth and differentiation of erythroid (BFU-E) and granulo-monocytic progenitors (CFU-GM). The base editing procedure did not impact on progenitors' viability (FIG. 3A). Compared to K562 cells, the editing profile in BFU-E pools showed the preferential targeting of one A in the different binding sites; however base editing efficiency was overall high, ranging between ˜60% to ˜90% (FIG. 3B). Indels were detected at high frequencies only in gRNA KO samples (83%) (FIG. 3C). The frequency of MCS-R2 deletion was ˜60% (FIG. 3D). RT-qPCR and Western Blot analyses showed a consistent α-globin down-regulation in BFU-E treated with either gRNA 2 or 4 (FIGS. 4A and B). It is noteworthy that the excessive α-globin down-regulation observed in gRNA KO samples by Western Blot and RP-HPLC (FIGS. 4B and C) generated the typical α-thalassemic phenotype characterized by the presence of Hb Bart and HbH tetramers composed of 4 γ- and 4 β-globin chains, respectively, as evaluated by CE-HPLC (FIG. 4C). On the contrary, base-edited samples showed a normal hemoglobin profile, demonstrating the safety of this therapeutic strategy (FIG. 4C).

### REFERENCES

Throughout this application, various references describe the state of the art to which this invention pertains. The disclosures of these references are hereby incorporated by reference into the present disclosure.
1. Taher, A. T., Weatherall, D. J. & Cappellini, M. D. Thalassaemia. Lancet Lond. Engl. 391, 155-167 (2018).
2. Kato, G. J. et al. Sickle cell disease. Nat. Rev. Dis. Primer 4, 18010 (2018).
3. Chandrakasan, S. & Malik, P. Gene therapy for hemoglobinopathies: the state of the field and the future. Hematol. Oncol. Clin. North Am. 28, 199-216 (2014).
4. Cavazzana, M., Antoniani, C. & Miccio, A. Gene Therapy for β-Hemoglobinopathies. Mol. Ther. J. Am. Soc. Gene Ther. 25, 1142-1154 (2017).
5. Sripichai, O. et al. Coinheritance of the different copy numbers of alpha-globin gene modifies severity of beta-thalassemia/Hb E disease. Ann. Hematol. 87, 375-379 (2008).
6. Embury, S. H. et al. Concurrent sickle-cell anemia and alpha-thalassemia: effect on severity of anemia. N. Engl. J. Med. 306, 270-274 (1982).
7. Higgs, D. R. et al. The interaction of alpha-thalassemia and homozygous sickle-cell disease. N. Engl. J. Med. 306, 1441-1446 (1982).
8. de Ceulaer, K. et al. alpha-Thalassemia reduces the hemolytic rate in homozygous sickle-cell disease. N. Engl. J. Med. 309, 189-190 (1983).
9. Mettananda, S., Gibbons, R. J. & Higgs, D. R. Understanding α-globin gene regulation and implications for the treatment of β-thalassemia. Ann. N. Y. Acad. Sci. 1368, 16-24 (2016).
10. Vernimmen, D. et al. Chromosome looping at the human alpha-globin locus is mediated via the major upstream regulatory element (HS-40). Blood 114, 4253-4260 (2009).
11. De Gobbi, M. et al. Tissue-specific histone modification and transcription factor binding in alpha globin gene expression. Blood 110, 4503-4510 (2007).
12. Mettananda, S. et al. Editing an α-globin enhancer in primary human hematopoietic stem cells as a treatment for β-thalassemia. Nat. Commun. 8, 424 (2017).
13. Milyavsky, M. et al. A distinctive DNA damage response in human hematopoietic stem cells reveals an apoptosis-independent role for p53 in self-renewal. Cell Stem Cell 7, 186-197 (2010).
14. Cromer, M. K. et al. Global Transcriptional Response to CRISPR/Cas9-AAV6-Based Genome Editing in CD34+ Hematopoietic Stem and Progenitor Cells. Mol. Ther. J. Am. Soc. Gene Ther. 26, 2431-2442 (2018).
15. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
16. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
17. Gaudelli, N. M. et al. Programmable base editing of A·T to G·C in genomic DNA without DNA cleavage. Nature 551, 464-471 (2017).
18. Rees, H. A. & Liu, D. R. Base editing: precision chemistry on the genome and transcriptome of living cells. Nat. Rev. Genet. 19, 770-788 (2018).
19. Yeh, W.-H., Chiang, H., Rees, H. A., Edge, A. S. B. & Liu, D. R. In vivo base editing of post-mitotic sensory cells. Nat. Commun. 9, 2184 (2018).
20. Pavani, G. et al. Ex vivo editing of human hematopoietic stem cells for erythroid expression of therapeutic proteins. Nat. Commun. 11, 3778 (2020).
21. Kluesner, M. G. et al. EditR: A Method to Quantify Base Editing from Sanger Sequencing. CRISPR J. 1, 239-250 (2018).
22. Brinkman, E. K., Chen, T., Amendola, M. & van Steensel, B. Easy quantitative assessment of genome editing by sequence trace decomposition. Nucleic Acids Res. 42, e168 (2014).

1. A method for repressing expression of α-globin in a eukaryotic cell comprising the step of contacting the eukaryotic cell with a gene editing platform comprising (a) at least one base-editing enzyme and (b) at least one guide RNA molecule for guiding the at least one base-editing enzyme to at least one target sequence in the MCS-R2 region present in the locus control region of HBA1 and HBA2 genes, thereby editing said MCS-R2 region and subsequently repressing the expression of α-globin in said eukaryotic cell.

2. The method of claim 1 wherein the gene editing platform is suitable for introducing some mutations in the MCS-R2 region so that at least one transcriptional activator binding site is disrupted in said region.

3. The method of claim 1 wherein the gene editing platform is suitable for disrupting at least one transcriptional activator binding site for GATA1 or NF-E2 in the MCS-R2 region.

4. The method of claim 1 wherein the gene editing platform is suitable for editing and disrupting the first, second, third and/or fourth GATA1 binding site(s) in the MCS-R2 region.

5. The method of claim 1 wherein the gene editing platform is suitable for editing and disrupting the first GATA1 binding site in the MCS-R2 region.

6. The method of claim 1 wherein the gene editing platform is suitable for editing and disrupting the second GATA1 binding site in the MCS-R2 region.

7. The method of claim 1 wherein the gene editing platform is suitable for editing and disrupting the third GATA1 binding site in the MCS-R2 region.

8. The method of claim 1 wherein the gene editing platform is suitable for editing and disrupting the fourth GATA1 binding site.

9. The method of claim 1 wherein the gene editing platform is suitable for editing and disrupting the first NF-E2 binding site.

10. The method of claim 1 wherein the eukaryotic cell is selected from the group consisting of hematopoietic progenitor cells, hematopoietic stem cells (HSCs), pluripotent cells and induced pluripotent stem cells (iPS).

11. The method of claim 1 wherein the at least one base-editing enzyme comprises a nickase.

12. The method of claim 11 wherein the nickase comprises the amino acid sequence as set forth in SEQ ID NO: 3 or SEQ ID NO:4.

13. The method of claim 1 wherein at least one base-editing enzyme is an adenosine deaminase.

14. The method of claim 13 wherein the adenosine deaminase is a TadA deaminase variant.

15. The method of claim 1 wherein the at least one base-editing enzyme is selected from the group consisting of ABE-NRCH, ABE-SpRY, ABE-NRTH, and ABEmax.

16. The method of claim 1 wherein the at least one base-editing enzyme and the corresponding at least one guide RNA molecule is chosen from Table B.

17. The method of claim 1 wherein the at least one guide RNA molecule comprises a plurality of guide RNA molecules that are designed for targeting a plurality of sequences in the MCS-R2 region.

18. The method of claim 1 wherein the at least one base-editing enzyme comprises a plurality of base-editing enzymes and the at least one guide RNA molecule comprises a plurality of guide RNA molecules that are designed for targeting a plurality of sequences in the MCS-R2 region.

19. A method for increasing fetal hemoglobin levels in a subject in need thereof, the method comprising transplanting a therapeutically effective amount of a population of eukaryotic cells obtained by the method of claim 1.

20. The method of claim 19 wherein the subject has been diagnosed with a hemoglobinopathy.

21. The method of claim 19 wherein different components of the gene editing platform are provided to the population of eukaryotic cells through the use of an RNA-encoded system.

22. A method of treating a β-hemoglobinopathy in a subject in need thereof, the method comprising transplanting a therapeutically effective amount of a population of eukaryotic cells obtained by the method of claim 1.

23. The method of claim 22 wherein the β-hemoglobinopathy is a sickle cell disease or β-thalassemia.

24. The method of claim 5 wherein the first GATA1 binding site is a TATC sequence in the MCS-R2 region and the method introduces one A>G conversion therein.

25. The method of claim 6 wherein the second GATA1 binding site is a GATA sequence in the MCS-R2 region and the method introduces at least one A>G conversion therein.

26. The method of claim 7 wherein the third GATA1 binding site is a GATT sequence in the MCS-R2 region, and the method introduces at least one A>G conversion therein.

27. The method of claim 8 wherein the fourth GATA1 binding site is a TATC sequence in the MCS-R2 region and the method introduces at least one A>G conversion therein.

28. The method of claim 9 wherein the first NF-E2 binding site is a TGACTCA sequence in the MCS-R2 region and the method introduces at least one A>G conversion therein.


SEQUENCE LISTING (36 sequences)

SEQ ID NO: 1 - DNA, 356 bp, Homo sapiens
  source 1..356: mol_type = other DNA
tcgaccctctggaacctatcagggaccacagtcagccaggcaagcacatctgcccaagccaagggtggaggcatgcagctgtgggggtctgtgaaaacacttgagggagcagataactgggccaaccatgactcagtgcttctggaggccaacaggactgctgagtcatcctgtgggggtggaggtgggacaagggaaaggggtgaatggtactgctgattacaacctctggtgctgcctccccctcctgtttatctgagagggaaggccatgcccaaagtgttcacagccaggcttcaggggcaaagcctgacccagacagtaaatacgttcttcatctggagctgaagaaattc

SEQ ID NO: 2 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, synthetic construct; identical to SEQ ID NO: 2
  REGION 1..1368: note = S. pyogenes nCas9 Protein Sequence having the D10A mutation

SEQ ID NO: 4 - protein, 1368 aa, synthetic construct
  REGION 1..1368: note = S. pyogenes nCas9 Protein Sequence having the H840A mutation
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 167 aa, Staphylococcus aureus
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 6 - protein, 5 aa, synthetic construct
  REGION 1..5: note = Linker
GGGGS

SEQ ID NO: 7 - protein, 5 aa, synthetic construct
  REGION 1..5: note = Linker
EAAAK

SEQ ID NO: 8 - protein, 16 aa, synthetic construct
  REGION 1..16: note = Linker
SGSETPGTSESATPES

SEQ ID NO: 9 - RNA, 20 nt, synthetic construct
  misc_feature 1..20: note = gRNA1
  source 1..20: mol_type = other RNA
acctatcagggaccacagtc

SEQ ID NO: 10 - RNA, 20 nt, synthetic construct
  misc_feature 1..20: note = gRNA2
  source 1..20: mol_type = other RNA
cagataactgggccaaccat

SEQ ID NO: 11 - RNA, 20 nt, synthetic construct
  misc_feature 1..20: note = gRNA3
  source 1..20: mol_type = other RNA
agttatctgctccctcaagt

SEQ ID NO: 12 - RNA, 20 nt, synthetic construct
  misc_feature 1..20: note = gRNA4
  source 1..20: mol_type = other RNA
ttgtaatcagcagtaccatt

SEQ ID NO: 13 - RNA, 20 nt, synthetic construct
  misc_feature 1..20: note = gRNA5
  source 1..20: mol_type = other RNA
ctcagataaacaggaggggg

SEQ ID NO: 14 - RNA, 20 nt, synthetic construct
  misc_feature 1..20: note = gRNA6
  source 1..20: mol_type = other RNA
ccatgactcagtgcttctgg

SEQ ID NO: 15 - DNA, 20 bp, synthetic construct
  misc_feature 1..20: note = Primer
  source 1..20: mol_type = other DNA
tggtcctgaaggatgagaag

SEQ ID NO: 16 - DNA, 20 bp, synthetic construct
  misc_feature 1..20: note = Primer
  source 1..20: mol_type = other DNA
agcaacagtcctttctctgg

SEQ ID NO: 17 - DNA, 21 bp, synthetic construct
  misc_feature 1..21: note = Primer
  source 1..21: mol_type = other DNA
cggtcaacttcaagctcctaa

SEQ ID NO: 18 - DNA, 20 bp, synthetic construct
  misc_feature 1..20: note = Primer
  source 1..20: mol_type = other DNA
acagaagccaggaacttgtc

SEQ ID NO: 19 - DNA, 18 bp, synthetic construct
  misc_feature 1..18: note = Primer
  source 1..18: mol_type = other DNA
gaaggtgaaggtcggagt

SEQ ID NO: 20 - DNA, 20 bp, synthetic construct
  misc_feature 1..20: note = Primer
  source 1..20: mol_type = other DNA
gaagatggtgatgggatttc

SEQ ID NO: 21-25 - skipped (5 entries)

SEQ ID NO: 26 - RNA, 20 nt, synthetic construct
  source 1..20: mol_type = other RNA
tgagtcatggttggcccagt

SEQ ID NO: 27 - RNA, 20 nt, synthetic construct
  source 1..20: mol_type = other RNA
tgctgattacaacctctggt

SEQ ID NO: 28 - RNA, 20 nt, synthetic construct
  source 1..20: mol_type = other RNA
gctgattacaacctctggtg

SEQ ID NO: 29 - DNA, 25 bp, synthetic construct
  source 1..25: mol_type = other DNA
ggcccagttatctgctccctcaagt

SEQ ID NO: 30 - DNA, 24 bp, synthetic construct
  source 1..24: mol_type = other DNA
aggcccatatctctgcccaagagc

SEQ ID NO: 31 - DNA, 27 bp, synthetic construct
  source 1..27: mol_type = other DNA
ggggacttttgccatgcctgaagtaga

SEQ ID NO: 32 - DNA, 27 bp, synthetic construct
  source 1..27: mol_type = other DNA
ggccccactccctgatcttaaccattt

SEQ ID NO: 33 - DNA, 22 bp, synthetic construct
  source 1..22: mol_type = other DNA
gcaaggtgaacgtggatgaagt

SEQ ID NO: 34 - DNA, 23 bp, synthetic construct
  source 1..23: mol_type = other DNA
taacagcatcaggagtggacaga

SEQ ID NO: 35 - DNA, 19 bp, synthetic construct
  source 1..19: mol_type = other DNA
cctgtcctctgcctctgcc

SEQ ID NO: 36 - DNA, 19 bp, synthetic construct
  source 1..19: mol_type = other DNA
ggattgccaaaacggtcac